(19) United States
(12) Patent Application Publication
(10) Pub. No.: US 2015/0040921 A1
(43) Pub. Date: Feb. 12, 2015

(54) Port Cover

(71) Applicant: Lori R. Craig, Germantown, MD (US)

(72) Inventor: Lori R. Craig, Germantown, MD (US)

(21) Appl. No.: 13/964,091

(22) Filed: Aug. 11, 2013

(51) Int. Cl. A61M 39/02

(52) U.S. Cl. CPC A61M 39/02; A61M 2039/0288; A61M 2039/0258; A61M 2039/0205
USPC 128/888

(57) ABSTRACT

The Port Cover is a soft, thin plastic covering that is used to cover the cream applied to the skin covering an implantable port, prior to treatment. The cream numbs the skin to minimize the pain from the needle insertion into the port when caregivers draw blood, administer treatments and therapies. FIG. 1 is the rounded rectangular version of the Port Cover; FIG. 2 is the perforated Port Cover. FIG. 3 is the circle design, FIG. 4 is the trapezoid design, and FIG. 5 is the square design. The varied designs will accommodate the need and preference of the patient. Currently, patients use products such as, saran wrap, plastic sandwich bags, wax paper, and paper towel to cover the numbing cream. The Port Cover is designed for single and duo ports, and is convenient to carry in a purse or wallet, prior to use.

### BACKGROUND OF INVENTION

The Port Cover was developed because of a need in the oncology and hematology medical industries. The Port Cover was designed for patients who are battling cancer or other blood disease disorders, requiring implantable ports. The Ports are placed in the upper right or left chest or arm areas to facilitate treatments, and to preserve veins due to long term treatments. The use of ports has become wide spread. The need for a product to cover the numbing cream applied to the skin covering the port is necessary. Patients forego using the numbing cream and deal with the uncomfortable needle stick, others use items like saran wrap, plastic lunch bags, wax paper etc., to cover the numbing cream. The Port Cover will provide a product that is designed specifically for the single and dual port designs, using a soft plastic that come in various shapes, and colors.

### BRIEF SUMMARY OF INVENTION

The Port Cover was designed to provide oncology and hematology patients who have implantable ports to receive medicines, treatments, therapies, blood transfusions or have blood drawn routinely, with a product that keeps the numbing cream used for treatments intact, minimizing the discomfort associated with the needle stick when the numbing cream is not used. The Port Cover is the alternative for patients who use nothing because they forgot to bring a covering for the numbing cream or those who use products like saran wrap, plastic sandwich bags, wax paper, paper towel, etc., to cover the numbing cream. The Port Cover designs are easy to use and can be carried in a purse, wallet, or briefcase. The Port Cover provides the comfort of having a product, specifically designed for the single or dual port designs.

### BRIEF DESCRIPTION OF THE SEVERAL VIEWS OF THE DRAWING

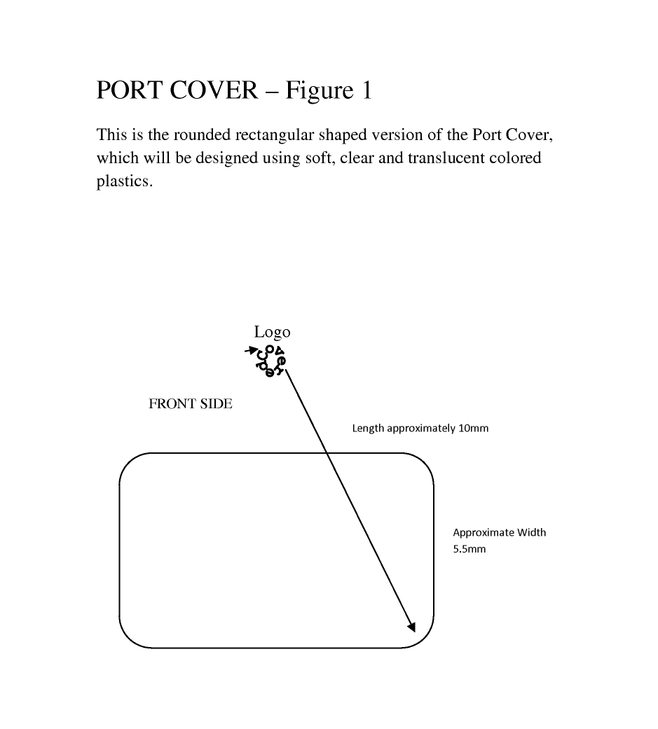

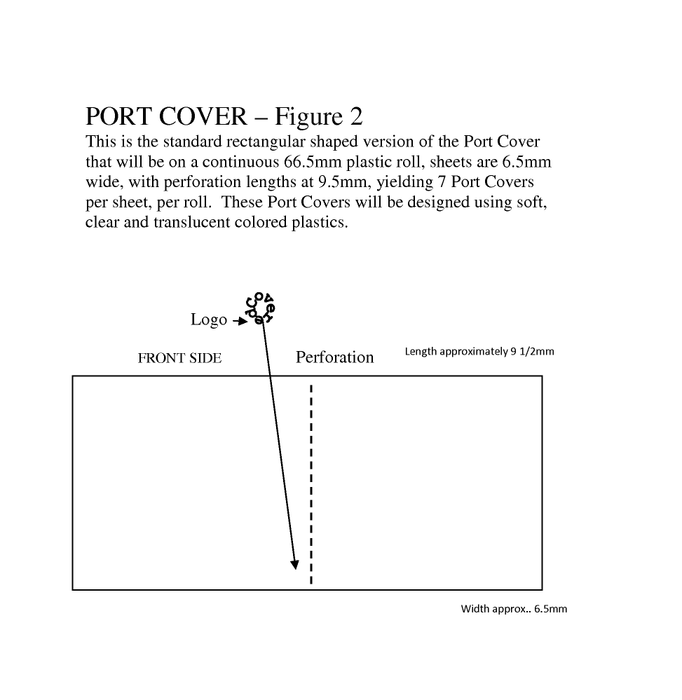

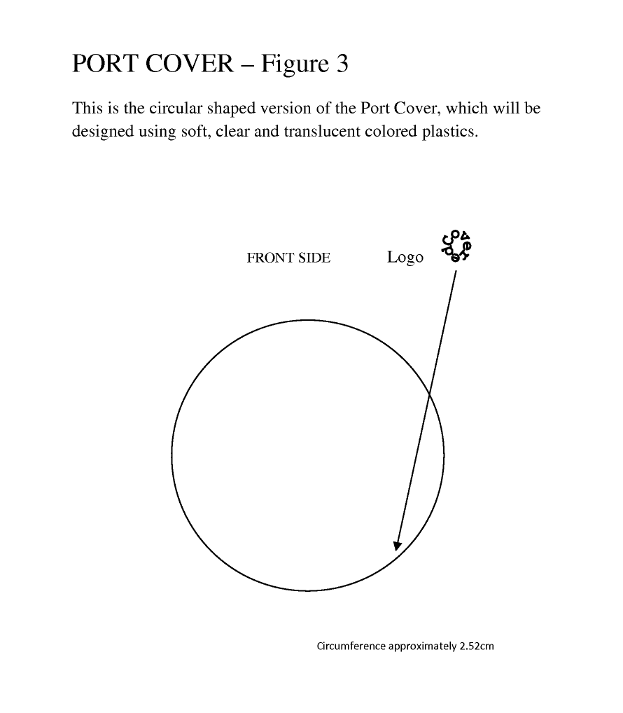

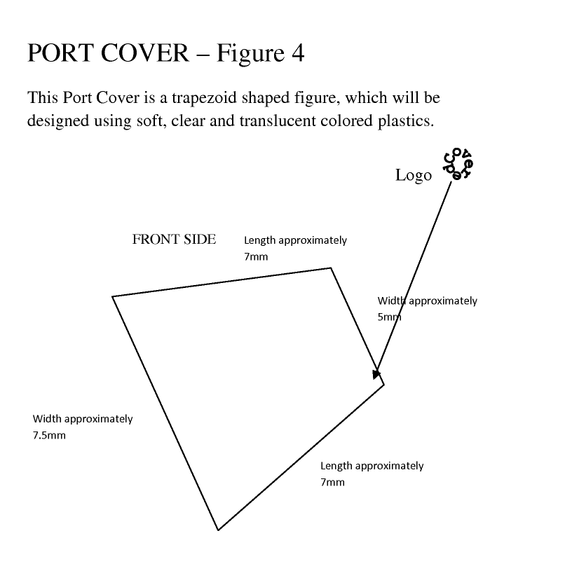

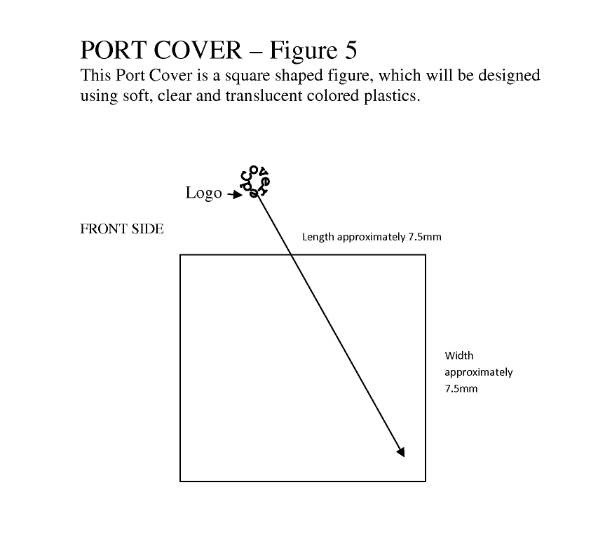

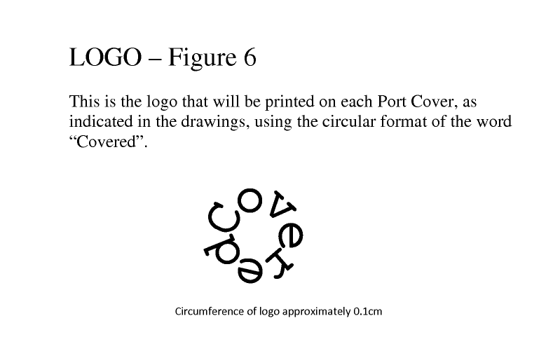

There are 6 Port Cover Drawings. The first drawing, Port Cover FIG. 1 is the rounded rectangular shaped version of the individual Port Cover. The second drawing FIG. 2 is the standard rectangular shaped design that will be on a plastic roll with perforation lengths at 9.5 mm. The third drawing FIG. 3 is the circular shape of the individual Port Cover. The fourth drawing FIG. 4 is a trapezoid version of the individual Port Cover view. The fifth drawing FIG. 5 is the square shaped view of the individual Port Cover, and the sixth drawing FIG. 6 is the “Covered” logo, which will be displayed in a circular format on each style of the Port Cover, FIG. 1 through FIG. 5. All Port Covers will use a soft plastic material and will come in clear and translucent colors.

### DETAILED DESCRIPTION OF THE INVENTION

The Port Cover is a soft plastic sheet that comes in various styles. It is used to cover the numbing cream applied to the skin covering an implantable port, prior to treatment. It minimizes the pain from the needle stick when caregivers draw blood, administer treatments and therapies. FIG. 1 is the rounded rectangular version of the Port Cover; FIG. 2 is the perforated Port Cover. FIG. 3 is the circle design, FIG. 4 is the trapezoid design, and FIG. 5 is the square design. Each design provides the coverage needed for both single and dual implantable ports. Each design will have the “covered” logo FIG. 6. The Port Covers is the alternative to using makeshift products like, saran wrap, plastic sandwich bags, wax paper, or paper towel, to cover the numbing cream. The Port Covers can be carried in a purse, wallet, or brief case, hassle free.

1. The Port Cover are thin, soft plastic sheets, that come in varying shapes and sizes as shown in FIGS. 1, 2, 3, 4 and 5. FIGS. 1, 3, 4, and 5 are single sheet designs; FIG. 2 is the perforated design.

2. The Port Covers are for patients who have implanted ports to facilitate multiple infusion therapies

3. The Port Covers of claim 1 conceals the numbing cream applied to the skin covering the port, before therapies.

4. The Port Covers of claim 3 keeps the numbing cream from transferring to clothing, preventing numbing of the skin.

5. The Port Covers of claim 1 are disposable and not reusable.

6. The Port Covers of claim 1 adheres to the numbing cream, without requiring adhesives.

7. The Port Cover logo FIG. 6 will be placed on each Port Cover where indicated in FIGS. 1, 2, 3, 4 and 5.
